(19) United States
(12) Patent Application Publication
(10) Pub. No.: US 2022/0009876 A1
(43) Pub. Date: Jan. 13, 2022

(54) METHOD FOR SYNTHESIZING D3 DOPAMINE RECEPTOR AGONISTS

(71) Applicant: POLYCORE THERAPEUTICS LLC, Philadelphia, PA (US)

(72) Inventors: Thais Motria SIELECKI-DZURDZ, Kennett Square, PA (US); Baoyu Ml, Wilmington, DE (US); Huiping ZHAO, East Brunsiwck, NJ (US); Sandhya KORTAGERE, Newton, PA (US)

(21) Appl. No.: 17/289,698

(22) PCT Filed: Oct. 31, 2019
(86) PCT No.: PCT/US2019/059036

§ 371 Date: Apr. 28, 2021

Related U.S. Application Data
(60) Provisional application No. 62/753,275, filed on Oct. 31, 2018.

(51) Int. Cl. C07C 209/62

(52) U.S. Cl. CPC C07C 209/62

(57) ABSTRACT

An improved method for synthesizing a compound according to formula (I) by reaction of a compound of formula (II) with a sulfinamide according to formula (III). The resultant compound is then reduced and hydrolyzed, and optionally alkylated or arylated to arrive at the compound according to formula (I).

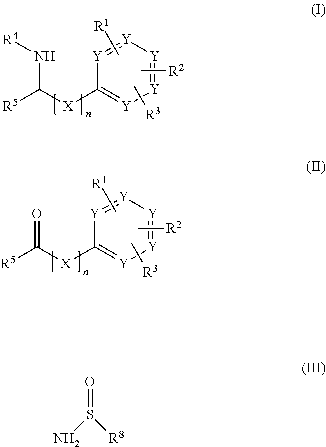

### BACKGROUND OF THE INVENTION

The instantly described invention is directed to improved methods for synthesizing D₃ dopamine receptor agonists.

D₃ dopamine receptor agonists, such as those produced by the processes described herein, have been found to be useful for treating or ameliorating symptoms of Parkinson's Disease. In particular, these dopamine agonists are depicted by the general formula (I):

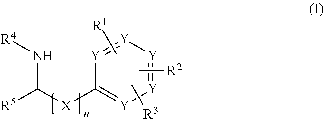

wherein:
R¹, R² and R³ are independently selected from the group consisting of H, cyano, hydroxyl, amino, acetamido, halo, alkoxy, nitro, C₁₋₆ alkyl, substituted C₁₋₆ alkyl, heteroalkyl, heterocyclyl, substituted heterocyclyl, aryl, substituted aryl, aryl-(C₁₋₃)alkyl, substituted aryl-(C₁₋₃)alkyl, carboxy, alkylcarboxy, formyl, alkyl-carbonyl, aryl-carbonyl, and heteroaryl-carbonyl;
R⁴ and R⁵ are independently selected from the group consisting of H, C₁₋₆ alkyl, substituted C₁₋₆ alkyl, heteroalkyl, heterocyclyl, substituted heterocyclyl, aryl, substituted aryl, aryl-(C₁₋₃)alkyl, and substituted aryl-(C₁₋₃)alkyl;
n is an integer from 2 to 8;
each X is independently O, C(R⁶)₂, N, or S, where R⁶ is H, cyano, hydroxyl, amino, acetamido, halo, alkoxy, nitro, C₁₋₆ alkyl, substituted C₁₋₆alkyl, heteroalkyl, heterocyclyl, substituted heterocyclyl, aryl, substituted aryl, aryl-(C₁₋₃)alkyl, substituted aryl-(C₁₋₃)alkyl, carboxy, alkylcarboxy, formyl, alkyl-carbonyl, aryl-carbonyl, and heteroaryl-carbonyl; and
each Y is independently O, C(R⁷), N or S, with at least three 2 Y being C(R⁷), where R⁷ is H, cyano, hydroxyl, amino, acetamido, halo, alkoxy, nitro, C₁₋₆ alkyl, substituted C₁₋₆ alkyl, heteroalkyl, heterocyclyl, substituted heterocyclyl, aryl, substituted aryl, aryl-(C₁₋₃)alkyl, substituted aryl-(C₁₋₃)alkyl, carboxy, alkylcarboxy, formyl, alkyl-carbonyl, aryl-carbonyl, and heteroaryl-carbonyl.

Previously known methods for synthesizing these compounds exhibited poor efficiency, thus leading to the desire for a method of synthesis which exhibited improved efficiency. The inventors have found these goals to be met with the method of synthesis identified herein.

### SUMMARY OF THE INVENTION

The instantly described invention is a method for producing compounds according to formula (I):

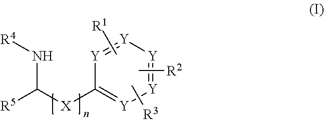

wherein:
R¹, R² and R³ are independently selected from the group consisting of H, cyano, hydroxyl, amino, acetamido, halo, alkoxy, nitro, C₁₋₆ alkyl, substituted C₁₋₆ alkyl, heteroalkyl, heterocyclyl, substituted heterocyclyl, aryl, substituted aryl, aryl-(C₁₋₃)alkyl, substituted aryl-(C₁₋₃)alkyl, carboxy, alkylcarboxy, formyl, alkyl-carbonyl, aryl-carbonyl, and heteroaryl-carbonyl;
R⁴ and R⁵ are independently selected from the group consisting of H, C₁₋₆ alkyl, substituted C₁₋₆ alkyl, heteroalkyl, heterocyclyl, substituted heterocyclyl, aryl, substituted aryl, aryl-(C₁₋₃)alkyl, and substituted aryl-(C₁₋₃)alkyl;
n is an integer from 2 to 8;
each X is independently O, C(R⁶)₂, N, or S, where R⁶ is H, cyano, hydroxyl, amino, acetamido, halo, alkoxy, nitro, C₁₋₆alkyl, substituted C₁₋₆ alkyl, heteroalkyl, heterocyclyl, substituted heterocyclyl, aryl, substituted aryl, aryl-(C₁₋₃)alkyl, substituted aryl-(C₁₋₃)alkyl, carboxy, alkylcarboxy, formyl, alkyl-carbonyl, aryl-carbonyl, and heteroaryl-carbonyl; and
each Y is independently O, C(R⁷), N or S, with at least three 2 Y being C(R⁷), where R⁷ is H, cyano, hydroxyl, amino, acetamido, halo, alkoxy, nitro, C₁₋₆ alkyl, substituted C₁₋₆ alkyl, heteroalkyl, heterocyclyl, substituted heterocyclyl, aryl, substituted aryl, aryl-(C₁₋₃)alkyl, substituted aryl-(C₁₋₃)alkyl, carboxy, alkylcarboxy, formyl, alkyl-carbonyl, aryl-carbonyl, and heteroaryl-carbonyl.

In the method described herein, a compound of formula (II)

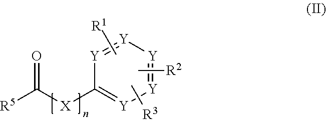

where R¹, R², R³, R⁵, n, X, and Y are as defined above; is reacted with a sulfinamide according to formula (III)

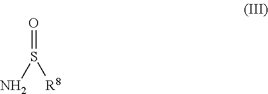

where R⁸ is optionally substituted C₁-C₆ alkyl or optionally substituted C₆-C₄ aryl or heteroaryl. As set forth in more detail below, by selecting the appropriate stereochemistry for the sulfinamide according to formula (III), it has been found to be possible to target particular stereochemistry for the desired dopamine receptor of formula (I) with a high degree of efficiency. The reaction of a compound of formula (II) with a sulfinamide of formula (III) results in a compound according to formula (IV)

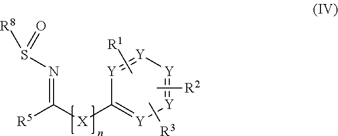

where R¹-R³, R⁵, R⁸, X, Y, and n are as defined above. This compound of formula (IV) is then condensed to form a compound of formula (V)

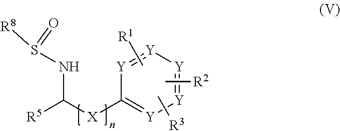

where R¹-R³, R⁵, R⁸, X, Y, and n are as defined above. The compound of formula (V) is then hydrolyzed to form a compound of formula (VI)

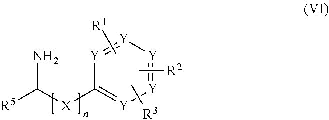

where R¹-R³, R⁵, X, Y, and n are as defined above. The compound of formula (VI) corresponds to the compound of formula (I) wherein R⁴ is H. In the event R⁴ is not hydrogen, the compound of formula (VI) may be reacted by alkylation or arylation to arrive at the compound according to formula (I).

### DETAILED DESCRIPTION

It was a goal of the instant invention to find an improved method for making of D₃ dopamine receptors such as those according to formula (I), in particular according to formula (IV). Previous methods for preparing these compounds showed poor efficiency in producing compounds of the desired stereochemistry. It was found, however, that by reacting with sulfinamides it was possible to more effectively produce compounds of a desired stereochemistry. In effect, by reacting with sulfinamides of particular stereochemistry, it was possible to more efficiently produce agonists with the desired stereochemistry.

In a first embodiment, the instantly described invention is a method for producing compounds according to formula (I):

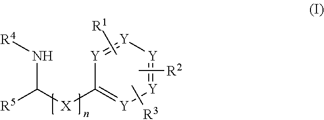

wherein:
R¹, R² and R³ are independently selected from the group consisting of H, cyano, hydroxyl, amino, acetamido, halo, alkoxy, nitro, C₁₋₆alkyl, substituted C₁₋₄alkyl, heteroalkyl, heterocyclyl, substituted heterocyclyl, aryl, substituted aryl, aryl-(C₁₋₃)alkyl, substituted aryl-(C₁₋₃)alkyl, carboxy, alkylcarboxy, formyl, alkyl-carbonyl, aryl-carbonyl, and heteroaryl-carbonyl; preferably, R¹, R², and R³ are independently H, hydroxyl, amino, or C₁₋₆ alkyl; in a particularly preferred embodiment, R¹, R², and R³ are each H;
R⁴ and R⁵ are independently selected from the group consisting of H, C₁₋₆ alkyl, substituted C₁₋₆ alkyl, heteroalkyl, heterocyclyl, substituted heterocyclyl, aryl, substituted aryl, aryl-(C₁₋₃)alkyl, and substituted aryl-(C₁₋₃)alkyl; in a particularly preferred embodiment, R⁴ is H;
n is an integer from 2 to 8; preferably n is 2, 3, 4, or 5; particularly preferably n is 2 or 3;
each X is independently O, C(R⁶)₂, N, or S, where R⁶ is H, cyano, hydroxyl, amino, acetamido, halo, alkoxy, nitro, C₁₋₆ alkyl, substituted C₁₋₆ alkyl, heteroalkyl, heterocyclyl, substituted heterocyclyl, aryl, substituted aryl, aryl-(C₁₋₃)alkyl, substituted aryl-(C₁₋₃)alkyl, carboxy, alkylcarboxy, formyl, alkyl-carbonyl, aryl-carbonyl, and heteroaryl-carbonyl; preferably X is in each case C(R⁶)₂ and particularly preferably X is in each case CH₂; and
each Y is independently O, C, N or S, with at least two Y being C; preferably each Y is independently C or N; particularly preferably all Y are C.

### A. Imination

In the method described herein, a compound of formula (II)

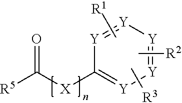

where R¹, R², R³, R⁵, n, X, and Y are as defined above; is reacted in an imination with a sulfinamide according to formula (III)

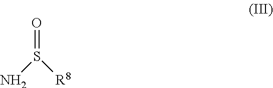

where R⁸ is optionally substituted C₁-C₆ alkyl or optionally substituted C₆-C₂₄ aryl or heteroaryl. R⁸ is preferably C₁₋₆ alkyl; particularly preferably R⁸ is tert-butyl.

The reaction with a sulfinamide is preferably carried out in the presence of a chiral tetra-substituted metal imination agent. Selection of such an imination agent would be according to the knowledge of the skilled artisan. It would be understood that any transition metal imination agent could be used. Metals in Groups 3-12 (IUPAC notation) could be used, with metals in Groups 3-11 preferred. Metals in Group 4 are particularly preferred. An exemplary imination agent would be Ti(R)₄, where R is optionally substituted alkyl or aryl. In a particularly preferred embodiment, the amination agent is Ti(R)₄ where R is isopropyl.

The reaction conditions for such an imination would be readily understood to the person of ordinary skill in the art. For example, such conditions can be seen in R. L. Reeves in S. Patai, Ed., The Chemistry of the Carbonyl Group, Interscience Publishers, London, 1966, p. 608-619; and J. K. Whitesell in B. M. Trost, et al, Ed., Comprehensive Organic Synthesis, Vol. 6, Pergamon Press, Oxford, 1991, p. 719. The contents of the cited pages are incorporated herein by reference for purposes of the amination conditions disclosed therein.

By selecting the appropriate stereochemistry for the sulfinamide according to formula (III), it has been found to be possible to target particular stereochemistry for the desired dopamine receptor of formula (I) with a high degree of efficiency. Thus, selecting of a sulfinamide in s-isomeric form would result in a product also in s-isomeric form to a high efficiency. The opposite would also be expected, where if the sulfinamide is utilized in r-isomeric form, the product would result in a product also in r-isomeric form to a high efficiency. This effect is further illustrated in the Examples presented below.

The reaction of a compound of formula (II) with a sulfinamide of formula (III) results in an imine compound according to formula (IV)

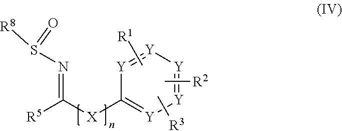

where R¹-R³, R⁵, R⁸, X, Y, and n are as defined above.

### B. Imine Reduction

This compound of formula (IV) is then reduced to form a compound of formula (V)

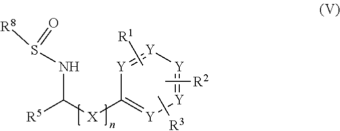

where R¹-R³, R⁵, R⁸, X, Y, and n are as defined above. Imine reduction conditions would be those known to the skilled artisan, and useful catalysts for imine reduction would also be known to the skilled artisans. Examples of known imine reduction conditions are set forth, for example, in A. V. Malkov, M. Figlus, S. Stončius, P. Kočovský, J. Org. Chem., 2007, 72, 1315-1325; Z. Wang, M. Cheng, P. Wu, S. Wei, J. Sun, Org. Lett., 2006, 8, 3045-3048; Z. Wang, X. Ye, S. Wei, P. Wu, A. Zhang, J. Sun, Org. Lett., 2006, 8, 999-1001; Y. Misumi, H. Seino, Y. Mizobe, J. Am. Chem. Soc., 2009, 131, 14636-14637; B. T. Cho, S. K. Kang, Tetrahedron, 2005, 61, 5725-5734; C.-H. Tien, M. R. Adams, M. J. Ferguson, E. R. Johnson, A. W. H. Speed, Org. Lett., 2017, 19, 5565-5568; A. Kaithal, B. Chatterjee, C. Gunanathan, J. Org. Chem., 2016, 81, 11153-11161; E. Selva, Y. Sempere, D. Ruiz-Martínez, O. Pablo, D. Guijarro, J. Org. Chem., 2017, 82, 13693-13699; T. Liu, L-y. Chen, Z. Sun, J. Org. Chem., 2015, 80, 11441-11446; B. W. Knettle, R. A. Flowers, II, Org. Lett., 2001, 3, 2321-2324; D. Li, Y. Zhang, G. Zhou, W. Guo, Synlett, 2008, 225-228; J. M. Blackwell, E. R. Sonmor, T. Scoccitti, W. E. Piers, Org. Lett., 2000, 2, 3921-3923; B. H. Lipshutz, H. Shimizu, Angew. Chem. Int. Ed., 2004, 43, 2228-2230; V. Khedkar, A. Tillak, M. Beller, Org. Lett., 2003, S, 4767-4770; G. Li, Y. Liang, J. C. Antilla, J. Am. Chem. Soc., 2007, 129, 5830-5831; and W. Wen, Y. Zeng, L.-Y. Peng, L.-N. Fu, Q.-X. Guo, Org. Lett., 2015, 17, 3922-3925. The contents of the cited pages are incorporated herein by reference for purposes of the imine reduction conditions disclosed therein.

Preferred imine reduction agents include HSiCl₃, H₂, NaHB₄, BH₃, and SmBr₂, NaBH₄ is a particularly preferred imine reduction agent.

While it is understood that the imination and imine reduction could be carried out in differing reactors, in an additional embodiment it is possible to perform the imination and imine reduction in a single reactor, termed a “one pot” synthesis.

### C. Hydrolysis

The compound of formula (V) is then hydrolyzed to form a compound of formula (VI)

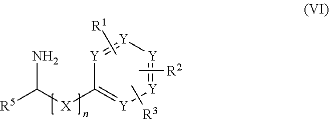

where R¹-R³, R⁵, X, Y, and n are as defined above. The compound of formula (VI) corresponds to the compound of formula (I) wherein R⁴ is H.

In the hydrolysis reaction, the sulfoxide group of formula (V) is removed. As noted above, depending on the stereochemistry of the sulfinamide used in the imination step, the resultant product would have a particular desired stereochemistry.

Reagents and conditions for performance of this hydrolysis step would be understood to the skilled artisan. Such hydrolysis reagents include, for example, bronsted acids, bronsted bases. These conditions are known, for example, from Remington's “Essentials of Pharmaceuticals,” 2013, at “Stability of Pharmaceutical Products,” p. 43-44, the contents of which are incorporated herein by reference for purposes of hydrolysis conditions disclosed therein. Preferred hydrolysis reagents are acids known to be useful. Particularly preferred is HCl.

The compound of Formula (VI) corresponds to the compound of Formula (I) where R⁴ is hydrogen. As noted above, the stereochemistry of the compound has been found to be determined based on the stereochemistry of the sulfinamide of formula (III) used in the imination step.

### D. Optional Alkylation/Arylation

In the event it is sought to produce a compound of formula (I) where R⁴ is not hydrogen, this may be obtained by a further alkylation/arylation step, the conditions of which would be known to the skilled artisan, utilizing reaction conditions and reagents known in the art. Such reactions are described, for example, in the following references, the contents of which are incorporated herein by reference for purpose of the alkylation/arylation conditions disclosed therein: March, Jerry (1985), Advanced Organic Chemistry: Reactions, Mechanisms, and Structure (3rd ed.), New York: Wiley, ISBN 0-471-85472-7: Organic Chemistry John McMurry 2nd Ed.; Organic Syntheses, Coll. Vol. 1, p. 48 (1941); Vol. 4, p. 3 (1925); Organic Syntheses, Coll. Vol. 1, p. 102 (1941); Vol. 8, p. 38 (1928); Organic Syntheses, Coll. Vol. 6, p. 104 (1988); Vol. 54, p. 58 (1974); Organic Syntheses, Coll. Vol. 6, p. 106 (1988); Vol. 54, p. 60 (1974); Organic Syntheses, Coll. Vol. 6, p. 75 (1988); Vol. 53, p. 13 (1973); Org. Synth, 2008, 85, 10-14; Organic Chemistry 4th Ed. Morrison & Boyd; J. F. Hartwig, “Organotransition Metal Chemistry: From Bonding to Catalysis” University Science Books, 2010. ISBN 978-1-891389-53-5; Karsten Eller, Erhard Henkes, Roland Rossbacher, Hartmut Höke “Amines, Aliphatic” in Ullmann's Encyclopedia of Industrial Chemistry, Wiley-VCH, Weinheim, 2005; Ervithayasuporn, V. (2012). “Synthesis and Reactivity of Nitrogen Nucleophiles-Induced Cage-Rearrangement Silsesquioxanes”. Inorg. Chem. 51 (22): 12266-12272.

In another embodiment of the instant invention, the compound of formula (I) corresponds to a compound of formula (VII)

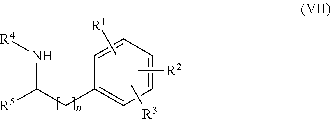

wherein:
R¹, R² and R³ are independently selected from the group consisting of H, cyano, hydroxyl, amino, acetamido, halo, alkoxy, nitro, C₁₋₆ alkyl, substituted C₁₋₆ alkyl, heteroalkyl, heterocyclyl, substituted heterocyclyl, aryl, substituted aryl, aryl-(C₁₋₃)alkyl, substituted aryl-(C₁₋₃)alkyl, carboxy, alkylcarboxy, formyl, alkyl-carbonyl, aryl-carbonyl, and heteroaryl-carbonyl; R⁴ and R⁵ are independently selected from the group consisting of H, C₁₋₆ alkyl, substituted C₁₋₆ alkyl, heteroalkyl, heterocyclyl, substituted heterocyclyl, aryl, substituted aryl, aryl-(C₁₋₃)alkyl, and substituted aryl-(C₁₋₃)alkyl; and
n is 2-8, in particular 2, 3, 4 or 5.

Compounds of formula (VII) are described, for example, in U.S. Pat. No. 9,675,565, the contents of which are incorporated herein by reference as to the disclosed compounds. These compounds have proven to be D₃ dopamine agonists which are effective in treating symptoms associated with Parkinson's Disease.

In the synthesis described herein, a compound of formula (VIII) is provided

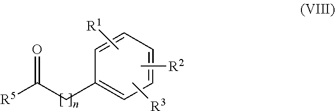

where R¹, R², R³, R⁵, and n are as defined above. This compound of formula (VIII) is reacted with a sulfinamide of formula (III) as defined above in an imination step. In a particularly preferred embodiment, the sulfinamide of formula (III) is tert-butyl sulfinamide, seen in formula (IX)

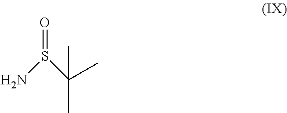

The use of s-tert-butyl sulfinamide would result in a final product in s-isomeric form with a high degree of efficiency. The use of r-tert-butyl sulfinamide would result in a final product in r-isomeric form with a high degree of efficiency. The imine reduction and hydrolysis would be carried out as described above.

In another embodiment, described herein is a process for making the S-isomer of 3-(2-chlorophenyl)-1-methyl-propylamine, according to formula (X)

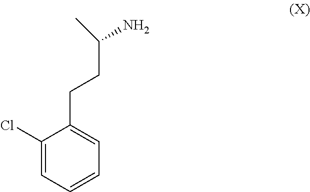

by reacting the compound according to formula (XI)

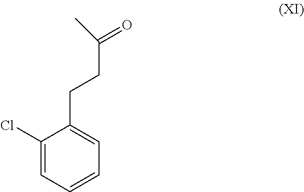

with (S)-tert-butylsulfinamide, resulting in a compound according to formula (XII)

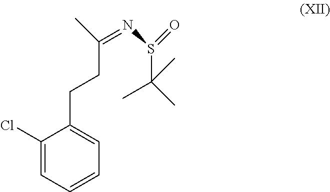 [SMILES: C/C(CCc1ccccc1Cl)=N/[S@@](=O)C(C)(C)C]

and then reducing the compound according to formula (XII), using the reagents discussed above, for example, NaBH₄, to arrive at the compound according to formula (XIII)

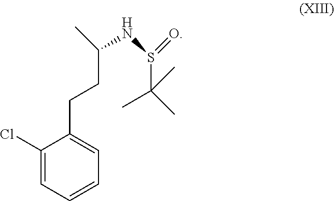 [SMILES: C[C@@H](CCc1ccccc1Cl)N[S@@](=O)C(C)(C)C]

The compound according to formula (XIII) is then hydrolyzed to form the compound according to formula (X).

In each of these embodiments, it is seen that the selection of a sulfinamide with a particular stereochemistry results in a compound with uniform stereochemistry. For example, the use of (S)-tert-butylsulfinamide results in a compound of Formula (XIII), which is a compound of either Formula (I) or Formula (IV) in the s-isomeric form.

In each of the embodiments described herein the compounds produced may be in the form given in Formula (I) or alternatively in any known pharmaceutically acceptable form, including, for example, salt form, for example in acid salt form.

### Exemplary Embodiments

In a first embodiment, a method for producing a compound according to formula (I):

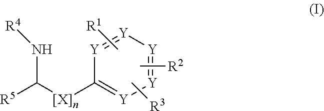

wherein:
R¹, R² and R³ are independently selected from the group consisting of H, cyano, hydroxyl, amino, acetamido, halo, alkoxy, nitro, C₁₋₆ alkyl, substituted C₁₋₆ alkyl, heteroalkyl, heterocyclyl, substituted heterocyclyl, aryl, substituted aryl, aryl-(C₁₋₃)alkyl, substituted aryl-(C₁₋₃)alkyl, carboxy, alkylcarboxy, formyl, alkyl-carbonyl, aryl-carbonyl, and heteroaryl-carbonyl;
R⁴ and R⁵ are independently selected from the group consisting of H, C₁₋₆ alkyl, substituted C₁₋₆ alkyl, heteroalkyl, heterocyclyl, substituted heterocyclyl, aryl, substituted aryl, aryl-(C₁₋₃)alkyl, and substituted aryl-(C₁₋₃)alkyl;
n is an integer from 2 to 8;
each X is independently O, C(R⁶)₂, N, or S, where R⁶ is H, cyano, hydroxyl, amino, acetamido, halo, alkoxy, nitro, C₁₋₆ alkyl, substituted C₁₋₆ alkyl, heteroalkyl, heterocyclyl, substituted heterocyclyl, aryl, substituted aryl, aryl-(C₁₋₃)alkyl, substituted aryl-(C₁₋₃)alkyl, carboxy, alkylcarboxy, formyl, alkyl-carbonyl, aryl-carbonyl, and heteroaryl-carbonyl; and
each Y is independently O, C(R⁷), N or S, with at least three 2 Y being C(R⁷), where R⁷ is H, cyano, hydroxyl, amino, acetamido, halo, alkoxy, nitro, C₁₋₆ alkyl, substituted C₁₋₆ alkyl, heteroalkyl, heterocyclyl, substituted heterocyclyl, aryl, substituted aryl, aryl-(C₁₋₃)alkyl, substituted aryl-(C₁₋₃)alkyl, carboxy, alkylcarboxy, formyl, alkyl-carbonyl, aryl-carbonyl, and heteroaryl-carbonyl; said method comprising:
a) reacting a compound of formula (II)

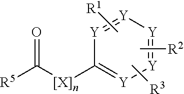

where R¹, R², R³, R⁵, n, X, and Y are as defined above; with a sulfinamide according to formula (III)

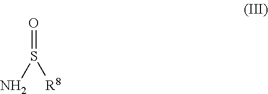

where R⁸ is optionally substituted C₁-C₆ alkyl or heteroalkyl or optionally substituted C₆-C₂₄ aryl or heteroaryl to form a compound of formula (IV)

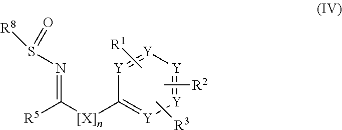

where R¹-R³, R⁵, R⁸, X, Y, and n are as defined above;
b) reducing the compound of formula (IV) to form a compound of formula (V)

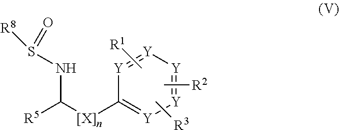

where R¹-R³, R⁵, R⁸, X, Y, and n are as defined above; and
c) hydrolyzing the compound of formula (V) and optionally alkylating or arylating to form the compound according to formula (I).

In a second embodiment, the method according to the first embodiment, wherein each Y is C, or N and each X is C(R⁶)₂ or N.

In a third embodiment, the method according to either the first or the second embodiment, wherein each Y is C and each X is C(R⁶)₂.

In a fourth embodiment, the method according to the third embodiment, wherein R⁶ is H.

In a fifth embodiment, the method according to any one of the first four embodiments, wherein no alkylation or arylation step is performed and the compound of formula (I) is a compound according to formula (VI)

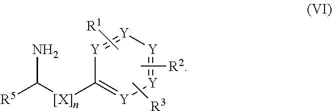

In a sixth embodiment, the method according to any one of the first five embodiments, wherein R⁸ is C₁-C₆ alkyl.

In a seventh embodiment, the method according to the sixth embodiment, wherein R⁸ is tert-butyl.

In an eighth embodiment, the method according to any of the first seven embodiments, wherein the sulfinamide according to formula (III) is s-tert-butylsulfinamide.

In a ninth embodiment, the method according to any of the first eight embodiments, wherein step a) is performed in the presence of an imination agent which is Ti(R)₄, where R is optionally substituted alkyl or aryl.

In a tenth embodiment, the method according to the ninth embodiment, wherein R is isopropyl.

In an eleventh embodiment, the method according to any one of the first ten embodiments, wherein step b) is performed with the aid of an imine reduction agent selected from the group consisting of HSiCl₃, H₂, NaBH₄, BH₃, and SmBr₂.

In a twelfth embodiment, the method according to the eleventh embodiment, wherein the imine reduction agent is NaBH₄.

In a thirteenth embodiment, the method according to any one of the first twelve embodiments, wherein the compound according to formula (I) is a compound according to formula (VII)

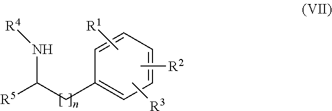

wherein:
R¹, R² and R³ are independently selected from the group consisting of H, cyano, hydroxyl, amino, acetamido, halo, alkoxy, nitro, C₁₋₆ alkyl, substituted C₁₋₆ alkyl, heteroalkyl, heterocyclyl, substituted heterocyclyl, aryl, substituted aryl, aryl-(C₁₋₃)alkyl, substituted aryl-(C₁₋₃)alkyl, carboxy, alkylcarboxy, formyl, alkyl-carbonyl, aryl-carbonyl, and heteroaryl-carbonyl;
R⁴ and R⁵ are independently selected from the group consisting of H, C₁₋₆ alkyl, substituted C₁₋₆ alkyl, heteroalkyl, heterocyclyl, substituted heterocyclyl, aryl, substituted aryl, aryl-(C₁₋₃)alkyl, and substituted aryl-(C₁₋₃)alkyl; and
n is 2-8; and wherein the compound according to formula (II) is a compound according to formula (VIII)

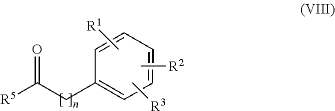

where R¹, R², R³, R⁵, and n are as defined above.

In a fourteenth embodiment, a method of synthesizing the S-isomer of 3-(2-chlorophenyl)-1-methyl-propylamine, according to formula (X)

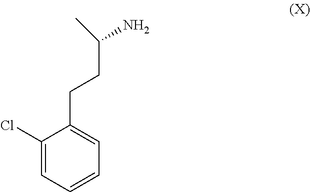

by reacting the compound according to formula (XI)

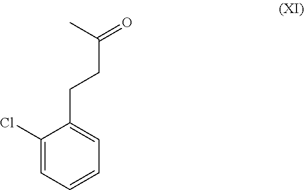

with (S)-tert-butylsulfinamide, resulting in a compound according to formula (XII)

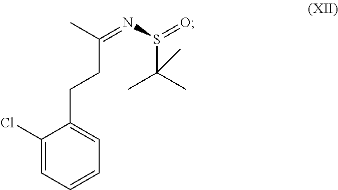 [SMILES: C/C(CCc1ccccc1Cl)=N/[S@@](=O)C(C)(C)C]

reducing the compound according to formula (XII) to arrive at the compound according to formula (XII)

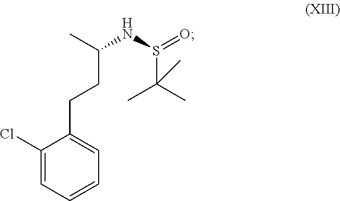 [SMILES: C[C@@H](CCc1ccccc1Cl)N[S@@](=O)C(C)(C)C]

and
hydrolyzing the compound according to formula (XIII) to arrive at the compound according to formula (X).

In a fifteenth embodiment, the method according to the fourteenth embodiment, wherein the reducing step is carried out in the presence of in imine reduction agent selected from the group consisting of HSiCl₃, H₂, NaBH₄, and SmBr₂.

In a sixteenth embodiment, the method according to the fifteenth embodiment, wherein the imine reduction agent is NaBH₄.

### Definitions

The term “alkyl”, by itself or as part of another substituent means, unless otherwise stated, a straight, or branched chain hydrocarbon having the number of carbon atoms designated (i.e. C₁-C₆ means one to six carbons) and includes straight, branched chain or cyclic groups. Examples include: methyl, ethyl, propyl, isopropyl, butyl, isobutyl, tert-butyl, pentyl, neopentyl, and hexyl. Most preferred is (C₁-C₆) alkyl, particularly ethyl, methyl and isopropyl.

The term “alkoxy” employed alone or in combination with other terms means, unless otherwise stated, an alkyl group, as defined above, connected to the rest of the molecule via an oxygen atom, such as, for example, methoxy, ethoxy, 1-propoxy, 2-propoxy (isopropoxy) and the higher homologs and isomers. The alkyl portion of the alkoxy group can have a designated number of carbon atoms as defined for alkyl groups above. Preferred are (C₁-C₃)alkoxy, particularly ethoxy and methoxy.

The term “carboxy” means —C(═O)—O-J, wherein J can be H, an inorganic or an organic counter ion, including an alkaline metal and a quaternary ammonium ion formed with an organic base, for example, trimethamine. For example, a carboxy includes a carboxylic acid —(C═O)—OH and metal carboxylate, such as —(C═O)—O⁻Na⁺.

The term “alkylamino” means —NH-alkyl, preferably —NH—(C₁-C₆)alkyl.

The term “acylamino” means —NH—(C═O)-alkyl, preferably

—NH—(C═O)—(C₁-C₆)alkyl.

The term “dialkyl amino” means —N[alkyl]2, preferably —N[(C₁-C₆)alkyl]₂.

The term “aroylamino” means —NH—(C═O)-aryl.

The term “carboxamido” means —(C═O)—NH₂.

The term “carbocyclic ring” refers to an cycloalkane ring formed by combining substituents attached to different carbon atoms. Preferably, R₄ and Q₂ can combine to form a cyclohexyl ring.

The terms “halo” or “halogen” by themselves or as part of another substituent mean, unless otherwise stated, a fluorine, chlorine, bromine, or iodine atom. Preferably, a halogen includes fluorine, chlorine, or bromine, more preferably, fluorine or chlorine.

The term “aromatic” refers to a carbocycle or heterocycle having one or more polyunsaturated rings having aromatic character (i.e. having (4n+2) delocalized □ (pi) electrons where n is an integer).

The term “aryl”, employed alone or in combination with other terms, means, unless otherwise stated, a carbocyclic aromatic system containing one or more rings (typically one, two or three rings) wherein such rings may be attached together in a pendent manner, such as a biphenyl, or may be fused, such as naphthalene. Examples include phenyl; anthracyl; and naphthyl. Preferred are phenyl and naphthyl, most preferred is phenyl.

“Substituted aryl” means an aryl, as defined above, substituted by one, two, three, four, or five substituents. In some embodiments, the substituents are selected from among the group consisting of halogen, fluoro; chloro; bromo; nitro; —NR₁₀R₁₁; aroylamino; cyano; carboxy; carboxamido; trifluoromethyl; —O—R₁₀; [—N(—R₁)—(CH₂)ₘ—C(—R₅)(—R₆)—(CH₂)ₙ—COOR₇]_{z}; [—N(—R₉)—(CH₂)ₘ—C(—R₅)(—R₆)—(CH₂)ₙ—COOR₇]_{z}; and C₁-C₁₀ saturated or unsaturated, straight or branched, cyclic or acyclic, chiral or achiral hydrocarbyl group, wherein optionally at least one carbon atom of the hydrocarbyl group is replaced by —N(—R₁)—, —O— or —S—. Preferably, a substituted aryl contains one to three substituents selected from methoxy, hydroxy, amino, and chloro, and fluoro, more preferably selected from amino, hydroxy, and methoxy.

The term “heterocycle” or “heterocyclyl” or “heterocyclic” by itself or as part of another substituent means, unless otherwise stated, an unsubstituted or substituted, stable, mono- or multi-cyclic heterocyclic ring system which consists of carbon atoms and at least one heteroatom selected from the group consisting of N, O, and S, and wherein the nitrogen and sulfur heteroatoms may be optionally oxidized, and the nitrogen atom may be optionally quaternized. The heterocyclic system may be attached, unless otherwise stated, at any heteroatom or carbon atom which affords a stable structure.

Examples of heterocyclyl (non-aromatic) include monocyclic groups such as: aziridinyl, oxiranyl, thiiranyl, azetidinyl, oxetanyl, thietanyl, pyrrolidinyl, pyrrolinyl, imidazolinyl, pyrazolidinyl, dioxolanyl, sulfolanyl, 2,3-dihydrofuranyl, 2,5-dihydrofuranyl, tetrahydrofuranyl, thiophanyl, piperidinyl, 1,2,3,6-tetrahydropyridinyl, 1,4-dihydropyridinyl, piperazinyl, morpholinyl, thiomorpholinyl, pyranyl, 2,3-dihydropyranyl, tetrahydropyranyl, 1,4-dioxanyl, 1,3-dioxanyl, homopiperazinyl, homopiperidinyl, 1,3-dioxepanyl, 4,7-dihydro-1,3-dioxepinyl and hexamethyleneoxidyl, preferably piperidinyl, piperazinyl and morpholinyl.

Examples of polycyclic heterocycles include: indolyl, particularly 3-, 4-, 5-, 6- and 7-indolyl, indolinyl, quinolyl, tetrahydroquinolyl, isoquinolyl, particularly 1- and 5-isoquinolyl, 1,2,3,4-tetrahydroisoquinolyl, cinnolinyl, quinoxalinyl, particularly 2- and 5-quinoxalinyl, quinazolinyl, phthalazinyl, 1,8-naphthyridinyl, 1,4-benzodioxanyl, coumarin, dihydrocoumarin, benzofuryl, particularly 3-, 4-, 1,5-naphthyridinyl, 5-, 6- and 7-benzofuryl, 2,3-dihydrobenzofuryl, 1,2-benzisoxazolyl, benzothienyl, particularly 3-, 4-, 5-, 6-, and 7-benzothienyl, benzoxazolyl, benzthiazolyl, particularly 2-benzothiazolyl and 5-benzothiazolyl, purinyl, benzimidazolyl, particularly 2-benzimidazolyl, benztriazolyl, thioxanthinyl, carbazolyl, carbolinyl, acridinyl, pyrrolizidinyl and quinolizidinyl.

The aforementioned listing of heterocyclyl and heteroaryl moieties is intended to be representative and not limiting.

“Substituted aryl” means an aryl, as defined above, substituted by one, two, three, four, or five substituents. In some embodiments, the substituents are selected from among the group consisting of halogen, fluoro; chloro; bromo; nitro; —NR₁₀R₁₁; aroylamino; cyano; carboxy; carboxamido; trifluoromethyl; —O—R₁₀; [—N(—R₁)—(CH₂)ₘ—C(—R₅)(—R₆)—(CH₂)ₙ—COOR₇]_{z}; [—N(—R₉)—(CH₂)ₘ—C(—R₅)(—R₆)—(CH₂)ₙ—COOR₇]_{z}; and C₁-C₁₀ saturated or unsaturated, straight or branched, cyclic or acyclic, chiral or achiral hydrocarbyl group, wherein optionally at least one carbon atom of the hydrocarbyl group is replaced by —N(—R₁)—, —O— or —S—. Preferably, a substituted aryl contains one to three substituents selected from methoxy, hydroxy, amino, and chloro, and fluoro, more preferably selected from amino, hydroxy, and methoxy.

All references disclosed herein are incorporated by reference. One skilled in the art will readily appreciate that the present invention is well adapted to carry out the objects and obtain the ends and advantages mentioned, as well as those inherent therein. The present invention may be embodied in other specific forms without departing from the spirit or essential attributes thereof and, accordingly, reference should be made to the appended claims, rather than to the foregoing specification, as indicating the scope of the invention.

### EXAMPLES

### A. Comparative Synthesis Route

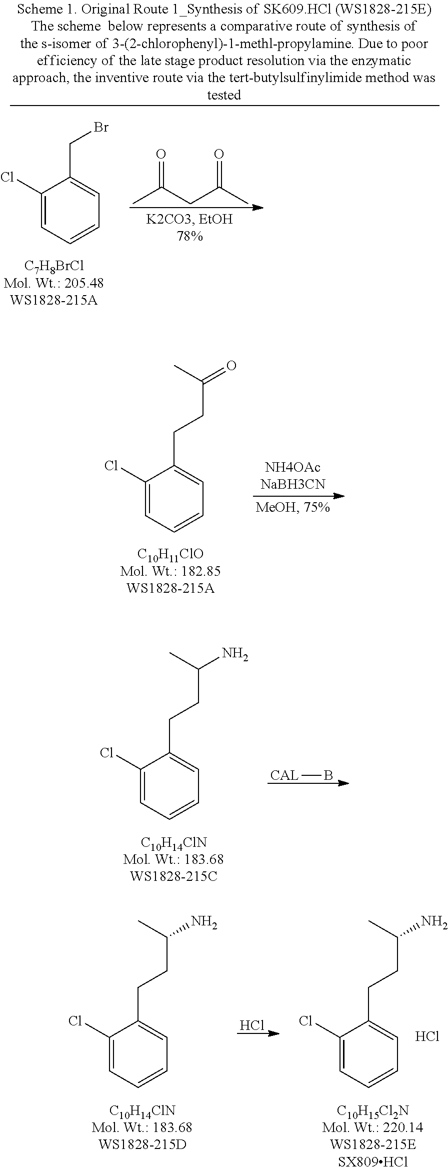

### B. Inventive Synthesis Route

The below scheme 2 represents the inventive route for synthesis tested herein.

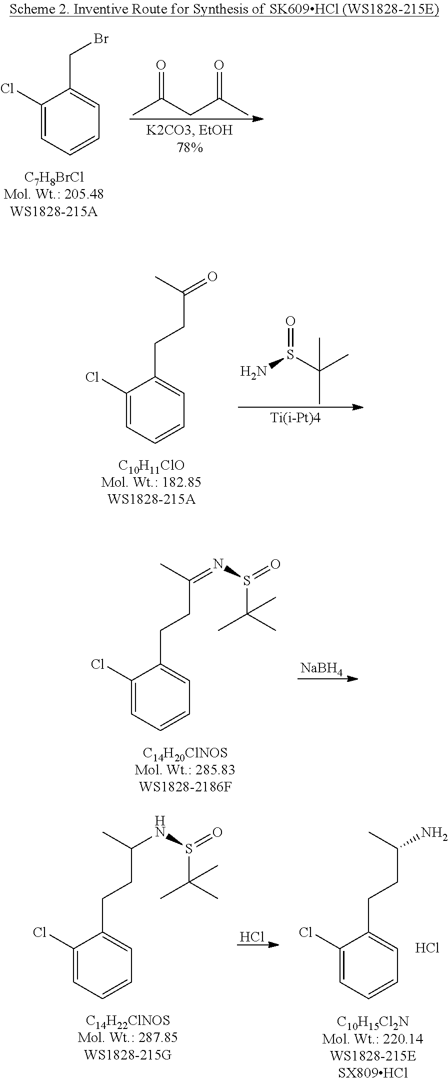

### C. Synthesis of WS1828-215B

For both the inventive and the comparative synthesis route, in an initial step compound WS 1828-215B was prepared. Initially, the reaction was thoroughly investigated and an optimized procedure was established. Ultimately experiments at 50 g, 200 g, and 150 g were carried out under optimized conditions and results are summarized in Table 1. Crude product was isolated and telescoped into the next stage without further purification.

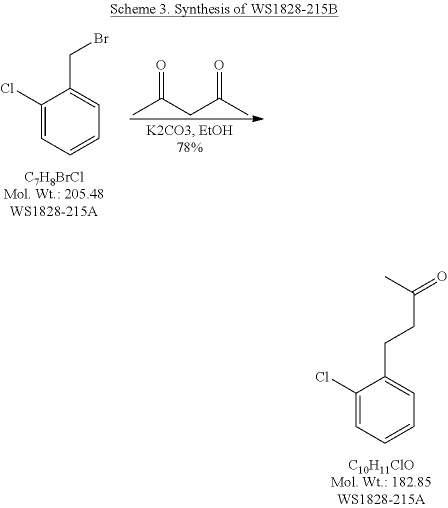

The specific procedure for producing WS1828-215B was as follows:

Charge 200 g of WS1828-215A (1.0 eq., 0.97 mol), 269 g of K₂CO₃ (2.0 eq., 1.95 mol), 2.68 g of 2, 4-pentanedione (2.0 eq., 1.95 mol) and 2.4 L of EtOH into a flask. The resultant mixture was agitated to dissolve and heated to reflux overnight.

The reaction mixture was concentrated, and the residue was diluted with 2.0 L of EtOAc and 2.0 L of water. Separated the phases, and the EtOAc layer was washed with 2.0 L, dried with 50 g of Na₂SO₄, filtered and concentrated to dryness.

The result: Wt.: 213 g, HPLC: 80.4%.

TABLE 1
Results for the synthesis of WS1828-215B

| | | | | | Product | |
|---|---|---|---|---|---|---|
| | | | | | % in | |
| | Input (g) | Eq. of | Eq. of | | RXN | Output/Crude |
| Experiment# | 215A | AcAc | K₂CO₃ | Solvent | Mixture | Yield/Purity |
| WS1138-149 | 5.0 | 1.1 | 1.1 | EtOH | NA | 0.6 g/13.5%/69.4% |
| WS1138-155 | 5.0 | 2.0 | 2.0 | EtOH | 84.7% | NA |
| WS1138-165 | 50 | 2.0 | 2.0 | EtOH | 80.9% | 47.2 g/106%/75.6% |
| WS1116-215 | 200 | 2.0 | 2.0 | EtOH | 83.6% | 213 g/120%/80.4% |
| WS1138-166 | 150 | 2.0 | 2.0 | EtOH | 80.6% | 220 g/165%/81.7% |

### D. Comparative Synthesis of WS1828-215C

As seen above, the comparative synthesis route differs from the inventive route in the steps after production of WS1828-215C. By this route, step 2 is reductive amination of WS1828-215B to form WS1828-215C. The procedure was briefly optimized and the quantities of NH₄OAc and NaBH₃CN were reduced to reasonable levels (6.0 and 2.5 eq. respectively) as summarized in Table 2.

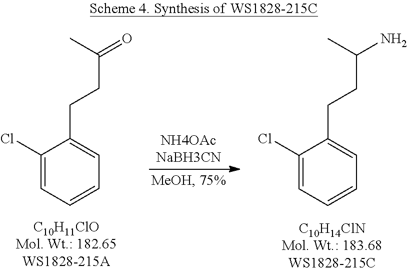

The specific procedure for producing WS1828-215 C was as follows:

8.8 g of WS1828-215B (1.0 eq., 48.6 mmol) and 15.0 g of NH4OAc (4.0 eq., 194.47 mmol) were dissolved in 89 mL of MeOH in a flask and cooled to 0° C. 4.58 g of NaBH₃CN (1.5 eq., 72.9 mmol) was added portionwise to the mixture. The resulting mixture was slowly warmed to 15-25° C. and stirred overnight.

The reaction mixture was concentrated and acidified to pH−1 with 89 mL of 2 N HCl, basified to pH˜14 with 178 mL of 3 N NaOH, and extracted with 3×266 mL of DCM. The combined DCM layer was concentrated to dryness and purified by column chromatography (silica wt.: 35 g, solvents: ethyl acetate-heptanes, gradient, 10/1 to 3:1).

The result: Wt.: 3.1 g. HPLC: 86.6%

TABLE 2
Results for the synthesis of WS1828-215C

| | | Eq. ratio of | PCT | | |
|---|---|---|---|---|---|
| | Input (g) | NH₄OAc/ | 215C/215B | | Yield |
| Entry | 215B | NaBH₃CN | (%) | Output/Purity | (A to C) |
| WS11138-153 | 0.43 | 12/5 | 99.7/0.3 | Original | Combined |
| WS11138-156 | 4.88 | 12/5 | 99.0/1.0 | procedure | yield: 58.1% |
| | | | | combined for | |
| | | | | work-up | |
| | | | | 3.1 g (87.8%) | |
| WS11138-161A | 4.1 | 4/1.5 | 98.1/1.9 | Combined | Combined |
| WS11138-161B | 4.1 | 6/2.5 | 98.7/1.3 | 4.0 g (90.2%) | yield: 54.6% |
| | | | | 0.5 g (82.8%) | |
| WS1116-217 | 8.8 | 4/1.5 | 98.0/2.0 | 3.1 g (86.6%) | 34.7% |

### E. Comparative Resolution of WS1828-215C

In the comparative route, WS1828-215 was put through an enzymatic resolution to target particular the desired s-isomeric form of the product. The results from a set of test experiments were not desirable due to poor product ee values (Table 3). Crystallization of the product by making the HCl salt failed to upgrade the ee to acceptable levels (still <90%).

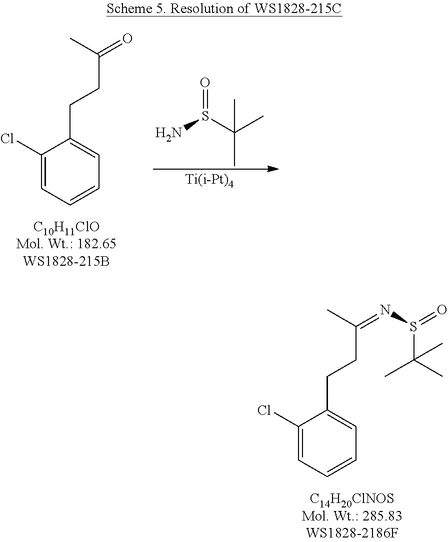

The specific procedure was as follows:

1.2 g of WS1828-215C (1.0 eq., 5.44 mmol), 0.72 g of Novozym435 was dissolved in 14.4 mL of EtOAc and stirred at 25˜30° C. for 14 hours. The reaction mixture was filtered and concentrated. The residue was diluted with 14.4 mL of MTBE and acidified to pH˜1 with 14.4 mL of 2 N HCl. Separated the phases, and the water layer was washed with 14.4 mL of MTBE. The water layer was basified with 3 N NaOH to pH˜14. Extracted the water layer with 2×14.4 mL of DCM. The combined organic layer was dried with 1.2 g of Na₂SO₄, filtered and concentrated to dryness.

The results: Wt.: 0.3 g, HPLC: 83.6%, ee: 86.4%

TABLE 3
Results for the resolution of WS1828-215C

| | Input(g) | Load of | | PCT | Output | | Purity | ee |
|---|---|---|---|---|---|---|---|---|
| Entry | 215C | Novozym435 | | (%) | (g) | | (%) | (%) |
| WS1138-159 | 1.0 g | 0.5 | g | 53.2/46.8 | 0.52 | g | 77.1% | 84.4% |
| WS1138-163 | 1.0 g | 0.1 | g | 52.1/47.9 | 0.45 | g | 79.7% | 73.5% |
| WS1116-213 | 1.2 g | 0.72 | g | 55.0/45.0 | 0.3 | g | 83.6% | 86.4% |

### F. Inventive Preparation of WS1828-215F

In the inventive procedure, the route requires reacting with a sulfinamide. In the example illustrated herein, the sulfinamide was tert-butylsulfinamide. The new route started with the condensation of WS1828-215B and (S)-tert-butanesulfinylamide to prepare WS1828-215F.

### Scheme 6. Synthesis of WS1828-215F

The specific procedure was as follows:

10 g of WS1828-215B (1.0 eq., 54.7 mmol), 7.96 g of (S)-tert-Butylsulfinamide (1.2 eq., 65.7 mmol), 31.1 g of Ti(OiP)₄ (2.0 eq., 110 mmol) and 150 mL of THF were charged to a flask, stirred and heated to reflux for 5 hours.

The mixture was cooled to r.t. and poured into 150 mL of brine. The resulting mixture was filtered through 20 g of Celite, the filter cake was washed with 20 mL of ethyl acetate. The filtrate was extracted with 150 mL of ethyl acetate. The combined organic layer was washed with 150 mL of brine, dried with 20 g of Na₂SO₄, filtered, concentrated. The residue was purified by column chromatography (Column condition: silica: 54 g, solvents: ethyl acetate/heptanes, gradient, 10/1 to 5/1).

The results:

Wt.: 7.0 g, HPLC: 95.8%.

TABLE 4
Results for preparation of WS1828-215F

| Exp# | Input (g) | Output (g) | Purity | Yield (A to F) |
|---|---|---|---|---|
| WS1116-219 | 2.0 | 1.9 | 94.6% | 60.7% |
| WS1116-223 | 10.0 | 7.0 | 95.8% | 44.7% |

### G. Inventive Preparation of WS1828-215G

According to the inventive route described herein, WS1828-215F was reduced by NaBH₄.

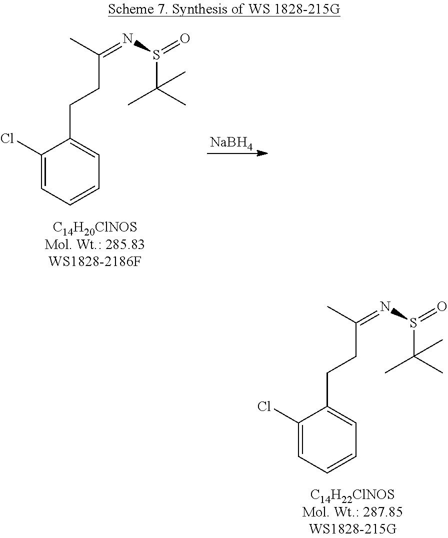 [SMILES: C/C(CCc1ccccc1Cl)=N/[S@@](=O)C(C)(C)C ; "NaBH4" ; C/C(CCc1ccccc1Cl)=N/[S@@](=O)C(C)(C)C]

The specific procedure was as follows:

Charged 7.0 g of WS1828-215F (1.0 eq., 24.5 mmol) and 70 mL of THF/H₂O (98/2) to a 100 mL RBF and cooled to −50° C. Charged 2.78 g of NaBH₄ (3.0 eq., 73.5 mmol) portion wise to the reaction mixture, then slowly warmed the mixture to 15-25° C. and stirred for 3 hours. The reaction mixture was concentrated. The residue was diluted with 70 mL of DCM, dried with 1 g of Na₂SO₄, filtered, concentrated and purified by column chromatography (silica wt.: 105 g, solvents: ethyl acetate-heptanes 5/1).

Result: Wt.: 3.4 g, HPLC: 96.5%.

TABLE 5
Results for Preparation of WS1828-215G

| | | | | | Purity | Purified | |
|---|---|---|---|---|---|---|---|
| Entry | Input(g) | Ratio of de | Output(g) | | (%) | de (%) | Yield (%) |
| WS1116-225 | 1.0 | 74.2/25.8 | 0.41 | g | 94.7% | 98.9% | 41% |
| WS1116-227 | 7.0 | 73.6/26.4 | 3.4 | g | 96.5% | 98.2% | 48.6% |

H. Alternative One Pot Inventive Preparation of WS1828-215G from WS1828-215B

As an alternative, it is possible to proceed from WS1828-215B to WS1828-215B in a one-pot synthesis method.

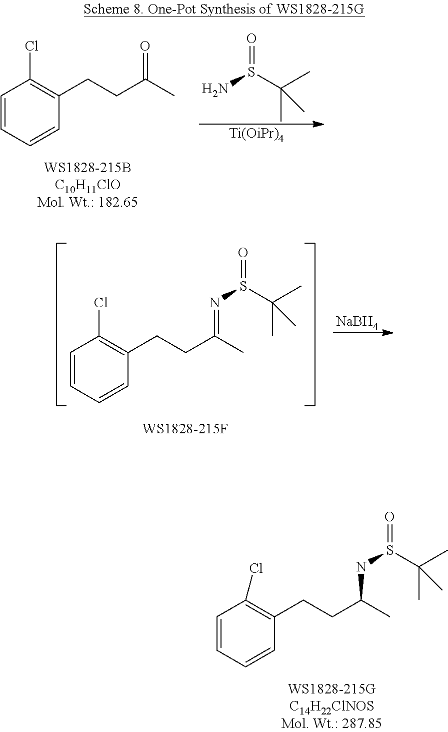

The procedure was as follows:

220 g of WS1828-215B (1.0 eq., 1.2 mol), 175 g of(S)-tert-butanesulfinamide (1.2 eq., 1.44 mol) and 685 g of Ti(OiP)₄ (2.0 eq., 2.4 mol) were charged to a 2 L RBF and heated to 60-65° C. for 3-5 hours. Charge 1100 mL of THE and 440 mL of EtOH to the mixture and cool the mixture to −10-0° C. Charge 50 g of NaBH₄ portion wise to the reaction mixture, then slowly warm the mixture to r.t. and stir for NLT 30 min. Charge 685 g of Celite and 4400 mL of EA to the mixture. Then charge 440 mL of water portionwise to the mixture and stir for NLT 30 min. Filter the mixture and wash the solid with 2200 mL of EA. Concentrate the filtrate and the washes to dryness. The residue was purified by column chromatography (silica: 6000 g, solvents: ethyl acetate/heptanes 5/1)

Results: Wt.: 75 g+65 g, HPLC: 94.4% and 91.3%

TABLE 6
One-Pot Preparation of WS1828-215G from WS1828-215B

| | | | | | | | | Yield |
|---|---|---|---|---|---|---|---|---|
| | | | | | | | Purified | (%) |
| Entry | Input(g) | | Ratio of de | Output(g) | | Purity (%) | de(%) | (A to G) |
| WS1138-167 | 5 | g | 80.1/19.9 | 4.6 | g | 91.0% | 92.7% | 29.2% |
| WS1138-169 | 5 | g | 79.0/21.0 | | | | | |
| WS1138-171 | 100 | g | 78.8/21.2 | 68 | g | 94.6% | 99.2% | 43.1% |
| WS1138-173 | 220 | g | 78.1/21.9 | 75 + 65 | g | 94.4% & | 97.7% & | 40.4% |
| | | | | | | 91.3% | 99.1% | |

### I. Inventive Route Preparation of WS1828-215E

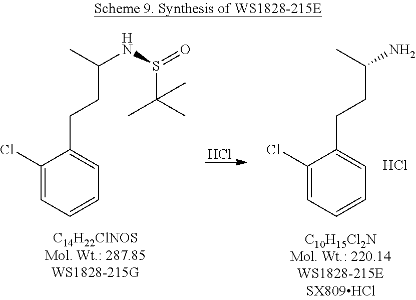 [SMILES: CC(CCc1ccccc1Cl)N[S@@](=O)C(C)(C)C ; Cl ; C[C@H](N)CCc1ccccc1Cl ; Cl]

To arrive at the final product, WS1828-215G was hydrolyzed as described below

The procedure was as follows:

120 g of WS1828-215G (1.0 eq., 0.417 mol) and 240 mL of MTBE were charged to a 1 L RBF. Charged 240 mL of 4 N HCl/IPAc to the RBF and stirred for 2 hours at 15-25° C. Filtered the mixture and washed the solid with 360 mL of MTBE. The product was dried under vacuum at 40-50° C. until constant weight.

Results: Wt.: 60.0 g, HPLC: 99.6%, ee: 99.6%.

TABLE 7
Results for inventive preparation of WS1828-215E

| Entry | Input(g) | Output(g) | Purity | ee | Yield |
|---|---|---|---|---|---|
| WS1138-176 | 10 | 5.1 | 99.2% | 99.6% | 66.9% |
| WS1138-177 | 10 | 3.7 | 99.6% | 99.6% | 48.2% |
| WS1138-178 | 120 | 60 | 99.6% | 99.6% | 65.4% |

As can be seen herein, the use of sulfinamide of a particular stereochemistry enabled synthesis of the product compound in the desired stereochemistry with a high level of purity not able to be achieved through the comparative route.

What is claimed is:

1. A method for producing a compound according to formula (I): 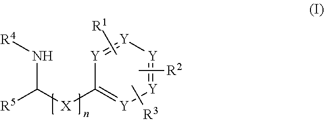
wherein:
R¹, R² and R³ are independently selected from the group consisting of H, cyano, hydroxyl, amino, acetamido, halo, alkoxy, nitro, C₁₋₆alkyl, substituted C₁₋₆alkyl, heteroalkyl, heterocyclyl, substituted heterocyclyl, aryl, substituted aryl, aryl-(C₁₋₃)alkyl, substituted aryl-(C₁₋₃)alkyl, carboxy, alkylcarboxy, formyl, alkyl-carbonyl, aryl-carbonyl, and heteroaryl-carbonyl;
R⁴ and R⁵ are independently selected from the group consisting of H, C₁₋₆ alkyl, substituted C₁₋₆ alkyl, heteroalkyl, heterocyclyl, substituted heterocyclyl, aryl, substituted aryl, aryl-(C₁₋₃)alkyl, and substituted aryl-(C₁₋₃)alkyl;
n is an integer from 2 to 8;
each X is independently O, C(R⁶)₂, N, or S, where R⁶ is H, cyano, hydroxyl, amino, acetamido, halo, alkoxy, nitro, C₁₋₆ alkyl, substituted C₁₋₆ alkyl, heteroalkyl, heterocyclyl, substituted heterocyclyl, aryl, substituted aryl, aryl-(C₁₋₃)alkyl, substituted aryl-(C₁₋₃)alkyl, carboxy, alkylcarboxy, formyl, alkyl-carbonyl, aryl-carbonyl, and heteroaryl-carbonyl; and
each Y is independently O, C(R⁷), N or S, with at least three 2 Y being C(R⁷), where R⁷ is H, cyano, hydroxyl, amino, acetamido, halo, alkoxy, nitro, C₁₋₆ alkyl, substituted C₁₋₆ alkyl, heteroalkyl, heterocyclyl, substituted heterocyclyl, aryl, substituted aryl, aryl-(C₁₋₃)alkyl, substituted aryl-(C₁₋₃)alkyl, carboxy, alkylcarboxy, formyl, alkyl-carbonyl, aryl-carbonyl, and heteroaryl-carbonyl; said method comprising:
a) reacting a compound of formula (II) 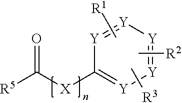
where R¹, R², R³, R⁵, n, X, and Y are as defined above; with a sulfinamide according to formula (III) 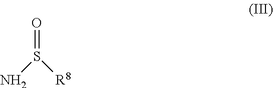
where R⁸ is optionally substituted C₁-C₆ alkyl or heteroalkyl or optionally substituted C₆-C₂₄ aryl or heteroaryl to form a compound of formula (IV) 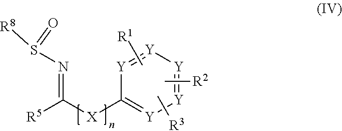
where R¹-R³, R⁵, R⁸, X, Y, and n are as defined above;
b) reducing the compound of formula (IV) to form a compound of formula (V) 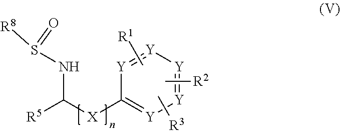
where R¹-R³, R⁵, R⁸, X, Y, and n are as defined above; and
c) hydrolyzing the compound of formula (V) and optionally alkylating or arylating to form the compound according to formula (I).

2. The method according to claim 1, wherein each Y is C, or N and each X is C(R⁶)₂ or N.

3. The method according to claim 1, wherein each Y is C and each X is C(R⁶)₂.

4. The method according to claim 3, wherein R⁶ is H.

5. The method according to claim 1, wherein no alkylation or arylation step is performed and the compound of formula (I) is a compound according to formula (VI)

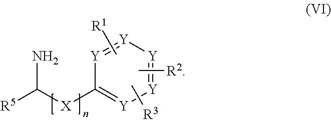

6. The method according to claim 1, wherein R⁸ is C₁-C₆ alkyl.

7. The method according to claim 6, wherein R⁸ is tert-butyl.

8. The method according to claim 1, wherein the sulfinamide according to formula (III) is s-tert-butylsulfinamide.

9. The method according to claim 1, wherein step a) is performed in the presence of an imination agent which is Ti(R)₄, where R is optionally substituted alkyl or aryl.

10. The method according to claim 9, wherein R is isopropyl.

11. The method according to claim 1, wherein step b) is performed with the aid of an imine reduction agent selected from the group consisting of HSiCl₃, H₂, NaBH₄, BH₃, and SmBr₂.

12. The method according to claim 11, wherein the imine reduction agent is NaBH₄.

13. The method according to claim 1, wherein the compound according to formula (I) is a compound according to formula (VII) 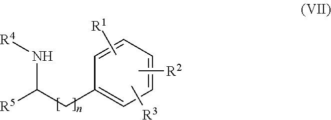
wherein:
R¹, R² and R³ are independently selected from the group consisting of H, cyano, hydroxyl, amino, acetamido, halo, alkoxy, nitro, C₁₋₆ alkyl, substituted C₁₋₆ alkyl, heteroalkyl, heterocyclyl, substituted heterocyclyl, aryl, substituted aryl, aryl-(C₁₋₃)alkyl, substituted aryl-(C₁₋₃)alkyl, carboxy, alkylcarboxy, formyl, alkyl-carbonyl, aryl-carbonyl, and heteroaryl-carbonyl;
R⁴ and R⁵ are independently selected from the group consisting of H, C₁₋₆ alkyl, substituted C₁₋₆ alkyl, heteroalkyl, heterocyclyl, substituted heterocyclyl, aryl, substituted aryl, aryl-(C₁₋₃)alkyl, and substituted aryl-(C₁₋₃)alkyl; and
n is 2-8; and wherein the compound according to formula (II) is a compound according to formula (VIII) 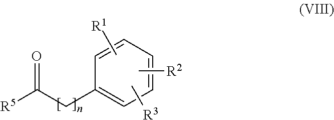
where R¹, R², R³, R⁵, and n are as defined above.

14. A method of synthesizing the S-isomer of 3-(2-chlorophenyl)-1-methyl-propylamine, according to formula (X) 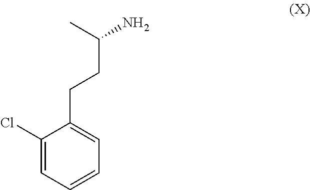
by reacting the compound according to formula (XI) 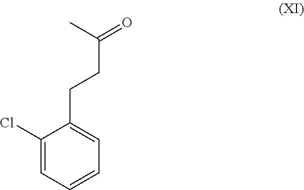
with (S)-tert-butylsulfinamide, resulting in a compound according to formula (XII) 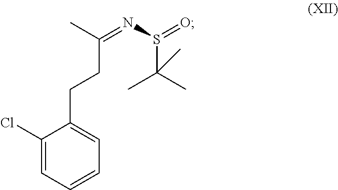 [SMILES: C/C(CCc1ccccc1Cl)=N/[S@@](=O)C(C)(C)C]
reducing the compound according to formula (XII) to arrive at the compound according to formula (XIII) 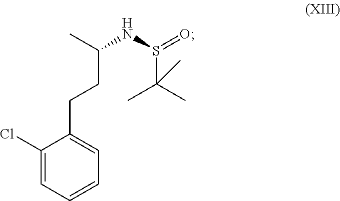 [SMILES: C[C@@H](CCc1ccccc1Cl)N[S@@](=O)C(C)(C)C]
and
hydrolyzing the compound according to formula (XIII) to arrive at the compound according to formula (X).

15. The method according to claim 14, wherein the reducing step is carried out in the presence of in imine reduction agent selected from the group consisting of HSiCl₃, H₂, NaBH₄, and SmBr₂.

16. The method according to claim 15, wherein the imine reduction agent is NaBH₄.
